(19) United States
(12) Patent Application Publication
(10) Pub. No.: US 2010/0063329 A1
(43) Pub. Date: Mar. 11, 2010

(54) METHOD FOR DECOMPOSING DI(PHENYLALKYL)PEROXIDES TO PRODUCE HYDROXYBENZENES AND PHENYLALKENES USING SOLID CATALYSTS

(76) Inventors: John Charles Saukaitis, Katy, TX (US); John Anthony Smegal, Houston, TX (US)

Correspondence Address: SHELL OIL COMPANY, P O BOX 2463, HOUSTON, TX, 772522463, US

(21) Appl. No.: 12/300,830

(22) PCT Filed: May 14, 2007
(86) PCT No.: PCT/US2007/068855

§ 371 Date: Apr. 22, 2009

Related U.S. Application Data
(60) Provisional application No. 60/747,356, filed on May 16, 2006.

(51) Int. Cl. C07C 39/00

(52) U.S. Cl. 568/716

(57) ABSTRACT

A method for decomposing di(phenylalkyl)peroxides to hydroxybenzenes and phenylalkene(s) using solid catalyst.

### FIELD OF THE INVENTION

The present application relates to a method for decomposing di(phenylalkyl)peroxides to produce hydroxybenzenes and phenylalkenes using solid catalyst.

### BACKGROUND OF THE INVENTION

Hydroxybenzenes are important organic chemicals with a wide variety of industrial uses. A number of processes are currently available for the production of hydroxybenzenes. One such process is known as the “cumene process.”

A cumene process begins with the production of cumene from benzene and propylene. The cumene is then oxidized to form cumene hydroperoxide:

C₆H₅C(CH₃)₂H+O₂→C₆H₅C(CH₃)₂OOH

The cumene hydroperoxide subsequently is cleaved into phenol and acetone:

C₆H₅C(CH₃)₂OOH→C₆H₅OH+(CH₃)₂CO.

The oxidation of other phenylalkyl hydroperoxides generally follows a similar pathway.

The process also generally produces a number of byproducts. In a cumene process, byproducts may include, for example, α-methylstyrene (AMS), acetophenone, dicumylperoxide (DCP), and dimethylbenzyl alcohol (DMBA). Less desirable byproducts include, for example, AMS dimer and cumyl phenol (CP).

On an industrial scale, cumene hydroperoxide typically is catalytically cleaved with dilute sulfuric acid. U.S. Pat. No. 5,463,136 (“Blackbourn”) describes a process in which “cumene hydroperoxide and sulfuric acid are reacted in a reflux cooled reactor, the products of which are transported under inhibited reaction conditions to a plug flow reactor, and are reacted to produce the phenol, acetone, and alpha-methylstyrene.” Abstract. Blackbourn explains that “the reflux cooled reactor products leaving the reactor . . . comprise between about 0.5% and 3.0% wt CHP. This insures that the CHP and DMBA reaction to DCP takes place and that only a very small percentage of AMS derived heavy ends are manufactured.” Blackbourn, col. 4, 11. 24-28. In the plug flow reactor, “the reactor utilizes high temperatures to drive the DCP and any remaining CHP to AMS, DMK [dimethyl ketone], and phenol.” Blackbourn, col. 4, 11. 15-17.

One disadvantage of using dilute sulfuric acid to cleave cumene hydroperoxide and to decompose DCP is that, in the presence of dilute sulfuric acid, DMBA tends to dehydrate to AMS. The AMS thus formed tends to form unwanted byproducts, including but not necessarily limited to AMS dimer and cumyl phenol (CP). Although it is possible to thermally crack AMS dimer and CP to produce AMS and phenol, yields are poor and a substantial amount of labor and equipment are required.

Efficient methods are needed to cleave phenylalkyl hydroperoxides and to decompose DCP to produce phenol at high yield while reducing yields of unwanted byproducts.

### SUMMARY OF THE INVENTION

The present application provides a method for decomposing di(phenylalkyl)peroxides using solid catalyst.

The present application also provides a method for both cleaving phenylalkyl hydroperoxides using solid catalyst and for decomposing di(phenylalkyl)peroxides using solid catalyst.

The present application also provides a multi-stage method for cleaving phenylalkyl hydroperoxides in one stage, and decomposing di(phenylalkyl)peroxides using solid catalyst in a different stage.

The present application also provides a multi-stage method for cleaving phenylalkyl hydroperoxides using solid catalyst in one stage, and decomposing di(phenylalkyl)peroxides using solid catalyst in a different stage.

The present application also provides a method for decomposing di(phenylalkyl)peroxide comprising feeding a decomposition feed comprising di(phenylalkyl)peroxide to a reactor containing solid decomposition catalyst under decomposition conditions which decompose the di(phenylalkyl)peroxide to produce hydroxybenzene and phenylalkene.

The present application also provides a method for decomposing di(phenylalkyl)peroxide comprising: subjecting a cleavage feed comprising phenylalkyl hydroperoxide in the presence of cleavage catalyst to cleavage conditions which cleave the phenylalkyl hydroperoxide and produce a cleavage product comprising the di(phenylalkyl)peroxide; and, subjecting a decomposition feed comprising the cleavage product to solid decomposition catalyst under decomposition conditions which decompose the di(phenylalkyl)peroxide and produce hydroxybenzene and phenylalkene.

The present application also provides a method for decomposing di(phenylalkyl)peroxide comprising: feeding a cleavage feed comprising phenylalkyl hydroperoxide to a first column packed with solid cleavage catalyst comprising a combination of oxidized metals and subjecting the cleavage feed to cleavage conditions which cleave the phenylalkyl hydroperoxide and produce a cleavage product comprising the di(phenylalkyl)peroxide; and, feeding the decomposition feed comprising the cleavage product to a second column packed with solid decomposition catalyst comprising a combination of oxidized metals under decomposition conditions which decompose the di(phenylalkyl)peroxide and produce hydroxybenzene and phenylalkene.

### BRIEF DESCRIPTION OF THE DRAWINGS

One or more embodiments of the invention are described in detail and by way of example with reference to the accompanying drawings.

FIG. 1 is a schematic diagram of an exemplary two stage reactor system for use in practicing the method, which is an adaptation of the bench scale system used in Examples 4-6.

FIG. 2 is a schematic diagram of another two stage reactor system suitable for practicing the method described herein.

### DETAILED DESCRIPTION OF THE INVENTION

The decomposition process of the present application cleaves di(phenylalkyl)peroxides having the following general structure:

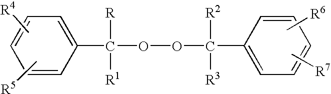

wherein
R, R¹, R², and R³ are independently selected from the group consisting of alkyl groups having 1 carbon atom or more, preferably having 5 carbon atoms or less, depending upon the phenylalkyl hydroperoxides cleaved, and a combination selected from the group consisting of (a) R and R¹ and (b) R² and R³ optionally may be linked to form a ring; and,
R⁴, R⁵, R⁶, and R⁷ independently are selected from the group consisting of hydrogen, hydroxyl groups, and methyl groups.

Examples of suitable di(phenylalkyl)peroxides include, but are not necessarily limited to dicumyl hydroperoxide, disec-butylbenzene hydroperoxide, dicyclohexylbenzene hydroperoxide, and combinations thereof.

In a cumene process, R⁴, R⁵, R⁶, and R⁷ are hydrogen and the di(phenylalkyl)peroxide is dicumyl peroxide, which has the following general structure:

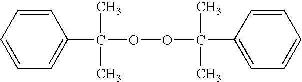

The di(phenylalkyl)peroxides may come from substantially any source. In a preferred embodiment, the di(phenylalkyl)peroxides are from the cleavage of phenylalkyl hydroperoxides having the following general structure:

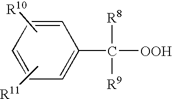

wherein
R⁸ and R⁹ independently are selected from the group consisting of alkylene groups having 1 or more carbon atom, preferably 5 carbon atoms or less, wherein R⁸ and R⁹ optionally may be linked to form a ring; and
R¹⁰ and R¹¹ independently are selected from the group consisting of hydrogen, hydroxyl groups, and alkyl groups having from about 1 to 4 carbon atoms. In one embodiment, R¹⁰ and R¹¹ are selected from the group consisting of hydrogen and methyl groups.

In preferred embodiments, R⁸ and R⁹ independently are selected from the group consisting of methyl groups and ethyl groups. In a cumene process, the phenylalkyl hydroperoxide is cumyl hydroperoxide. In a sec-butyl benzene process, the phenylalkyl hydroperoxide is sec-butylbenzene hydroperoxide. In a cyclohexylbenzene hydroperoxide process, the hydroperoxide is cyclohexylbenzene hydroperoxide. In one embodiment, the hydroperoxide is a combination of cumyl hydroperoxide and sec-butylbenzene hydroperoxide.

The choice of R¹⁰ and R¹¹ will depend upon the desired product. During oxidation of phenylalkanes, R¹⁰ and R¹¹ groups having 2 carbon atoms or more would be expected to oxidize. Depending upon the oxidation conditions, R¹⁰ and R¹¹ groups having 2 carbon atoms or more could attain various levels of oxidation. Upon substantially complete oxidation, R¹⁰ and R¹¹ groups having 2 carbon atoms or more could oxidize to the corresponding hydroperoxides and be cleaved to hydroxybenzene and the corresponding ketones. Under differing oxidation conditions, R¹⁰ and R¹¹ groups having 2 carbon atoms or more could oxidize to a hydroxybenzene group and acetaldehyde. Oxidation is less likely to occur where the substituent is a methyl group. In one embodiment, R¹⁰ and R¹¹ are hydrogen.

Cleavage of the phenylalkyl hydroperoxides produces a cleavage product comprising ketones and hydroxybenzenes. The cleavage product also includes byproducts, including but not necessarily limited to phenylalkenes, phenylalkyl ketones, di(phenylalkyl) peroxides, and phenylalkyl alcohols. Depending upon the conditions, the cleavage product also may comprise phenylalkene-derived heavy ends. Examples of phenylalkene-derived heavy ends include dimers of phenylalkene and/or the reaction product between phenylalkene and phenol.

Ketones in the cleavage product generally have the following structure:

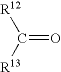

wherein the R¹² and R¹³ are alkyl groups having 1 or more carbon atoms, preferably having 6 carbon atoms or less. R¹² and R¹³ also may be joined to form a ring. In one embodiment, R¹² and R¹³ are joined to form cyclohexanone.

Hydroxybenzenes in the cleavage product generally have the following structure:

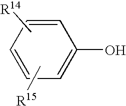

wherein R¹⁴ and R¹⁵ independently are selected from the group consisting of hydrogen, hydroxyl groups, and methyl groups.

Phenylalkenes in the cleavage product have the following general structure:

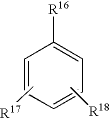

wherein
R¹⁶ is an alkenyl group comprising one or more unsaturated carbon-carbon bonds and having 2 carbon atoms or more, preferably having 6 carbon atoms or less, depending upon the phenylalkyl hydroperoxides cleaved; and,
R¹⁷ and R¹⁸ independently are selected from the group consisting of hydrogen, hydroxyl groups, and methyl groups. In one embodiment, R¹⁷ and R¹⁸ independently are selected from the group consisting of hydrogen and methyl groups.

Where the process is a cumene process, R¹⁷ and R¹⁸ are hydrogens, and the phenylalkene is α-methylstyrene (AMS), which has the following general structure:

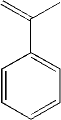

Phenylalkyl ketones which may be byproducts in the cleavage product generally have the following structure:

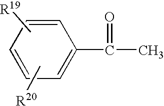

wherein R¹⁹ and R²⁰ independently are selected from the group consisting of hydrogen, hydroxyl groups, and methyl groups.

Where the process is a cumene process, the phenylalkyl ketone generally is acetophenone, which has the following structure:

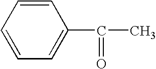

The phenylalkyl alcohols in the cleavage product generally have the following structure:

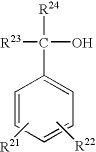

wherein
R²¹ and R²² independently are selected from the group consisting of hydrogen, hydroxyl groups, and methyl groups; and,
R²³ and R²⁴ independently are selected from the group consisting of alkyl groups having 1 to 6 carbon atoms, preferably 1 to 2 carbon atoms, depending upon the phenylalkyl hydroperoxides cleaved.

Where the process is a cumene process, R²¹ and R²² are hydrogen and the phenylalkyl alcohol is dimethylbenzyl alcohol (DMBA), which has the following structure:

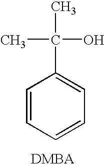

Where the process is a sec-butylbenzene process, R²¹ and R²² are hydrogen and the phenylalkyl alcohol is ethylmethyl benzyl alcohol (EMBA). EMBA has the following general structure:

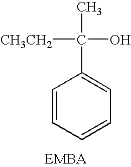

### The Decomposition Process

In one aspect, the application provides a method for decomposing di(phenylalkyl)peroxides using solid catalyst, or catalyst which is present as a solid under the particular process conditions.

Given the teachings in the present application, persons of ordinary skill in the art will be able to optimize the decomposition conditions using a given solid catalyst to accomplish a number of things, namely:
(a) to convert 80 wt. % or more of the di(phenylalkyl)peroxides present in the decomposition feed to hydroxybenzene and phenylalkene;
(b) to produce a decomposition product comprising a final concentration of di(phenylalkyl)peroxide of from about 0.05 wt. % to about 0.1 wt. %, based on the total weight of the decomposition product; and
(c) to produce a selectivity to α-methyl styrene of about 0.70 or more, based on the components in the decomposition feed that can theoretically produce AMS. In an advantageous embodiment, the decomposition conditions may be optimized to produce selectivity to α-methyl styrene of 0.80 or more, based on the components in the decomposition feed that can theoretically produce AMS.

The use of solid catalyst has a number of other advantages. Advantages include, but are not necessarily limited to minimizing handling of hazardous liquid acids; eliminating a neutralization step; reducing the water content of the neutralized reaction solution; reducing the energy cost of boiling additional water; removing corrosive salts from the reaction mixture; increasing the yield of desired products; minimizing impurities; reducing equipment costs required to run the process; reducing operating costs; and, minimizing unwanted hydroxyketones and ketone condensation products.

The decomposition feed may be any feed comprising about 5 wt. % or less di(phenylalkyl)peroxide based on the total weight of the decomposition feed. A preferred decomposition feed is the cleavage product from cleaving phenylalkyl hydroperoxides, which generally comprises hydroxybenzene and ketone. The decomposition feed suitably comprises about 0.3 wt. % or more, more preferably about 0.5 wt. % or more, preferably up to about to about 5 wt. %, more preferably up to about 3 wt. % di(phenylalkyl)peroxide, based on the total weight of the decomposition feed.

The decomposition process may occur as a single stage in a single reactor or may be part of a multiple stage process using a single reactor or multiple reactors. In one embodiment, the decomposition process is a multiple stage process comprising (a) cleavage of phenylalkyl hydroperoxides to produce a cleavage product in a first stage, and (b) decomposition of the cleavage product comprising di(phenylalkyl)peroxide in a second stage.

In one embodiment, the cleavage of phenylalkyl hydroperoxide and the decomposition of di(phenylalkyl)peroxide occurs in the same reactor, and the cleavage catalyst is the same as the decomposition catalyst. In another embodiment, the cleavage and decomposition occur in different reactors, and the cleavage catalyst and the decomposition catalyst are the same or different catalysts. In one embodiment using multiple reactors, the cleavage catalyst and the decomposition catalyst are both solid catalysts. In an advantageous embodiment, using multiple reactors, the cleavage catalyst and the decomposition catalyst are both solid catalysts comprising a combination of oxidized metals.

It is desirable for the decomposition conditions to maximize selectivity to phenylalkene and to maximize the conversion of di(phenylalkyl)peroxides to hydroxybenzene and phenylalkenes. In a cumene process, it also is desirable for the decomposition conditions to maximize selectivity to α-methyl styrene (AMS).

In a commercial cumene process using dilute sulfuric acid as catalyst, acceptable selectivity to α-methyl styrene (AMS) is about 0.55 or more, based on the components in the cleavage feed (cumene hydroperoxide feed) that can theoretically produce AMS. Advantageous commercial cumene processes using dilute sulfuric acid can produce selectivity to α-methyl styrene of about 0.70 or more, based on the components in the cleavage feed that can theoretically produce AMS.

Given the teachings in the present application, persons of ordinary skill in the art will be able to optimize the decomposition conditions using a given catalyst to produce a decomposition product comprising a final concentration of di(phenylalkyl)peroxide of from about 0.05 wt. % to about 0.1 wt. %, based on the total weight of the decomposition product. Generally, the decomposition conditions may be optimized to convert 80 wt. % or more of the di(phenylalkyl)peroxides present in the decomposition feed to hydroxybenzene and phenylalkene. In an advantageous embodiment, the decomposition conditions are optimized to convert 90 wt. % or more, suitably 95 wt. % or more, suitably up to 100 wt. % of the di(phenylalkyl)peroxides present in the decomposition feed to hydroxybenzene and phenylalkene.

Given the teachings in the present application, persons of ordinary skill in the art will be able to achieve the foregoing rates of conversion of di(phenylalkyl)peroxide while producing a selectivity to α-methyl styrene of about 0.70 or more, based on the components in the decomposition feed that can theoretically produce AMS. In one embodiment, the decomposition conditions are optimized to produce selectivity to α-methyl styrene of 0.73 or more. In an advantageous embodiment, the decomposition conditions are optimized to produce selectivity to α-methyl styrene of 0.80 or more, based on the components in the decomposition feed that can theoretically produce AMS.

In one embodiment, the decomposition conditions comprise a bottom column temperature of 90° C. or greater. In one embodiment, the decomposition conditions comprise a bottom column temperature of greater than 90° C. In one embodiment, the decomposition conditions comprise a bottom column temperature of from about 90° C. to about 170° C. In an advantageous embodiment, the decomposition conditions comprise a bottom column temperature of from about 90° C. to about 126° C. In one embodiment, the decomposition conditions comprise a bottom column temperature of from about 110° C. to about 120° C. In yet another embodiment, the decomposition conditions comprise a bottom column temperature of from about 115° C. to about 117° C.

In one embodiment, the decomposition conditions comprise a pressure of about 15 kPa to 8000 kPa. In one embodiment, the decomposition conditions comprise a pressure of from about 55 kPa to 7000 kPa. In one embodiment, the decomposition pressure is atmospheric pressure (typically about 100 kPa).

In one embodiment, the decomposition feed rate is 1 gram or less of decomposition feed per gram of catalyst per hour. In one embodiment, the decomposition feed rate is less than 1 gram of decomposition feed per gram of catalyst per hour. In one embodiment, the decomposition feed rate is from about 0.3 gram to about 1 gram of decomposition feed per gram of catalyst per hour. In an advantageous embodiment, the decomposition feed rate is 0.6 gram or less of decomposition feed per gram of catalyst per hour. In another advantageous embodiment, the decomposition feed rate is 0.45 gram or less of decomposition feed per gram of catalyst per hour. In yet another advantageous embodiment, the decomposition feed rate is 0.3 gram or less of decomposition feed per gram of catalyst per hour.

The process may be conducted continuously or batchwise. In one embodiment of a continuous process, the LHSV is from about 0.1 to 100 hr⁻¹, preferably from about 20 to about 60 hr⁻¹, based on the concentration of di(phenylalkyl)peroxide(s). If the reaction is batchwise, then the residence time is from about 1 to about 360 minutes, preferably from about 1 to about 180 minutes.

Under the conditions and with the catalysts used in the Examples, lowering the decomposition feed rate produced higher selectivity to α-methyl styrene and lower yields of dicumylperoxide. Specifically, Examples 4 and 5 demonstrate that, at bottom column temperatures of from 115-117° C. and using a decomposition feed containing about 3 wt. % or less (specifically 2.71 wt. %) dicumylperoxide, the decomposition feed rate could be adjusted to produce a selectivity to α-methyl styrene of 0.80-0.82, based on the components in the decomposition feed that could theoretically produce AMS. Under the same conditions, 80 wt. % or more of the dicumylperoxide in the decomposition feed was converted to α-methyl styrene and phenol. The final decomposition product contained 0.5 wt. % or less dicumylperoxide, based on the total weight of the decomposition product.

### The Cleavage Process

In one embodiment, the decomposition feed is the cleavage product from cleaving one or more phenylalkyl hydroperoxides. As used herein the plural form of a word, such as “hydroperoxides,” generally may be interpreted as singular or plural. In one embodiment, the cleavage of phenylalkyl hydroperoxides is performed in the presence of solid catalyst to produce the cleavage product.

The process of cleaving the phenylalkyl hydroperoxide may be batchwise or continuous. In one embodiment, the process is continuous. As used herein, the word “continuous” or “continuously” is intended to include processes which are continuous, but which may be subject to interruptions for various practical reasons. Examples of such interruptions include maintenance of equipment, cleaning, updating of equipment, and the like. By way of example only, and without limiting the process to a specific amount of downtime, the downtime in a “continuous” process generally is about 5% or less, based on total available operating time.

The cleavage feed comprises a phenylalkyl hydroperoxide feed comprising one or more phenylalkyl hydroperoxides. By way of example only, the cleavage feed comprises from about 0.5 wt. % to about 3 wt. % water, based on the total weight of the cleavage feed.

The cleavage feed may have substantially any concentration of phenylalkyl hydroperoxides. However, in order to avoid the need to handle large quantities of diluent, the phenylalkyl hydroperoxide feed suitably comprises 70 wt. % or more phenylalkyl hydroperoxides. In one embodiment, the phenylalkyl hydroperoxide feed comprises more than 70 wt. % phenylalkyl hydroperoxides, based on the total weight of the cleavage feed. In another embodiment, the phenylalkyl hydroperoxide feed comprises from about 70 wt. % to about 90 wt. % phenylalkyl hydroperoxides. In yet another embodiment, the phenylalkyl hydroperoxide feed comprises 80 wt. % or more phenylalkyl hydroperoxide. In one embodiment, the phenylalkyl hydroperoxide feed comprises from about 80 wt. % to about 88 wt. % phenylalkyl hydroperoxide, based on the total weight of the cleavage feed.

The cleavage feed is subjected to cleavage conditions effective to produce a cleavage product comprising di(phenylalkyl)peroxide. The cleavage process may involve the use of any cleavage catalyst. Examples of suitable cleavage catalysts include solid catalysts and mineral acids. Substantially any solid catalyst effective to cleave the di(phenylalkyl)peroxide may be used. In one embodiment, the cleavage catalyst is solid catalyst comprising a combination of oxidized metals.

In this embodiment, the cleavage reaction mixture, comprising a ketone feed and the phenylalkyl hydroperoxide feed, is subjected to cleavage conditions in the presence of solid catalyst. The reactor may be packed with solid catalyst, or the solid catalyst may be fed to the reactor. In one embodiment, the solid catalyst is fed to the reactor with the cleavage feed. The amount of solid catalyst is sufficient to catalyze the reaction. The reactor may be packed with catalyst, or the catalyst may be fed to the reactor. In one embodiment, the catalyst is fed to the reactor with the cleavage feed.

In one embodiment of a continuous process, the phenylalkyl hydroperoxide feed rate is 1 gram or less of phenylalkyl hydroperoxide feed per gram of catalyst per hour. Where the reaction is continuous, the cleavage conditions comprise a liquid hourly space velocity (LHSV) of from about 0.1 hr⁻¹ to 100 hr⁻¹, preferably from about 20 hr⁻¹ to about 60 hr⁻¹, based on the concentration of phenylalkyl hydroperoxide. Where the reaction is batchwise, the cleavage conditions comprise a residence time of from about 1 minute to about 360 minutes, preferably from about 1 minute to about 180 minutes.

An additional ketone feed is not required. However, feeding a ketone feed to the cleavage reactor will aid in reducing the production of non-recoverable by-products, for example, phenylalkyl alcohols and phenylalkene dimers. In one embodiment, the cleavage feed also comprises ketone feed.

Where a ketone feed is provided, the ketone feed rate is from about 0.1 wt. % to about 10 wt. % based on the phenylalkyl hydroperoxide feed rate. In one embodiment, the ketone feed rate is about 10 wt. % based on the phenylalkyl hydroperoxide feed rate. In other words, where the phenylalkyl hydroperoxide feed rate is 1 gram or less of phenylalkyl hydroperoxide feed per gram of catalyst per hour, the ketone feed rate is from about 0.001 gram to about 0.1 gram or less of ketone feed per gram of catalyst per hour. In one embodiment, the ketone feed rate is about 0.1 gram of ketone feed per gram of catalyst per hour.

The one or more ketones in the ketone feed suitably are the same as the ketones produced by cleaving the phenylalkyl hydroperoxides. In one embodiment, the ketone feed is recycled from a cleavage product separation zone.

The cleavage conditions comprise subjecting the cleavage reaction mixture to a temperature of 90° C. or less, preferably about 40° C. or more, more preferably about 50° C. or more, more preferably from about 50° C. to about 70° C. The temperature may be maintained in any suitable manner.

The cleavage conditions also comprise subjecting the cleavage reaction mixture to a pressure of from about 15 kPa to 8000 kPa, more preferably from atmospheric pressure to from about 55 kPa to 7000 Kpa. In one embodiment, the pressure is atmospheric pressure (typically about 100 kPa).

A variety of reactor types are suitable, including, for example, packed bed reactors, fluidized bed reactors, slurry reactors, continuous stirred tank reactors (CSTR's), reflux cooled (boiling) reactors, reactive distillation columns, plug-flow reactors (“PFR's”), and plug-flow reactors with recycle (PFRR's).

Optimum cleavage conditions may be established by adjusting various parameters. The conditions will vary with the number of reactors and the type of reactor(s) used. For example, where the reactor is a packed bed reactor, the temperature and catalyst bed size may be varied to achieve a maximum yield of phenol or an optimum slate of phenol and desired byproducts, including di(phenylalkyl)peroxide and/or α-methyl styrene (AMS). Persons of ordinary skill in the art will be able to establish optimum conditions using a particular reactor system, catalyst, and feed.

The selectivity of the process to various components may vary depending upon a number of factors, including whether the process is batchwise or continuous, and the intended use of the cleavage reaction mixture.

Where the cleavage product is designed to feed to a second stage in which di(phenylalkyl)peroxide is cleaved, the relatively mild cleavage conditions increase the concentration of di(phenylalkyl)peroxide in the cleavage product relative to the amount of di(phenylalkyl)peroxide in the cleavage feed. In a preferred embodiment, the cleavage product comprises from about 1 wt. % to about 5 wt. % di(phenylalkyl)peroxide.

The cleavage conditions also advantageously produce about 2 wt. % or less phenylalkene, preferably 0.5 wt. % or less phenylalkene, based on the total weight of the cleavage product, excluding ketone feed. The cleavage catalyst and the cleavage conditions also preferably maintain or reduce the concentration of phenylalkyl ketone(s). In a preferred embodiment, the cleavage catalyst and the cleavage conditions produce an amount of phenylalkenes and phenylalkene-derived heavy ends, such as dimers, of about 2 wt. % or less, preferably about 0.5 wt. % or less, based on the total weight of the cleavage product.

A portion of the cleavage product preferably is recycled to the cleavage reactor. Preferably, the ratio of recycled cleavage product to cleavage feed is from about 10:1 to about 100:1 on a weight basis, and more preferably from about 20:1 to 40:1 on a weight basis. In one embodiment, from about 10 wt. % to about 40% wt. of the cleavage product is recycled to the cleavage reactor. In one embodiment, from about 20% wt. to about 30% wt. of the cleavage product is recycled to the cleavage reactor. More preferably about 20% wt. of the cleavage product is recycled to the cleavage reactor.

The cleavage product in a cumene process typically comprises from about 0.5 wt. % to about 3.0 wt. % cumene hydroperoxide and from about 1 wt. % to about 5 wt. % of dicumylperoxide, based on the total weight of the cleavage product.

### Exemplary Reactor Systems

The type of reactor(s) used will vary depending upon whether the process is a single stage process or a multiple stage process. A variety of reactor types are suitable, including packed bed reactors, continuous stirred tank reactors (CSTR's), reflux cooled (boiling) reactors, reactive distillation columns, plug-flow reactors (“PFR's”), and plug-flow reactors with recycle (PFRR's). In one embodiment of a multi-stage process performed in multiple reactors, the cleavage reactor is a continuous stirred tank reactor or a reactive distillation column, and the second reactor is a plug flow reactor. In another embodiment of a multi-stage process performed in multiple reactors, two packed bed columns are used. Where the solid catalyst comprises pillared clay, preferred reactors are selected from the group consisting of packed bed reactors, fluidized bed reactors, and slurry reactors.

One embodiment for single or multi-stage processes uses the system described in U.S. Patent Application Publication No. 2004/0236152, incorporated herein by reference.

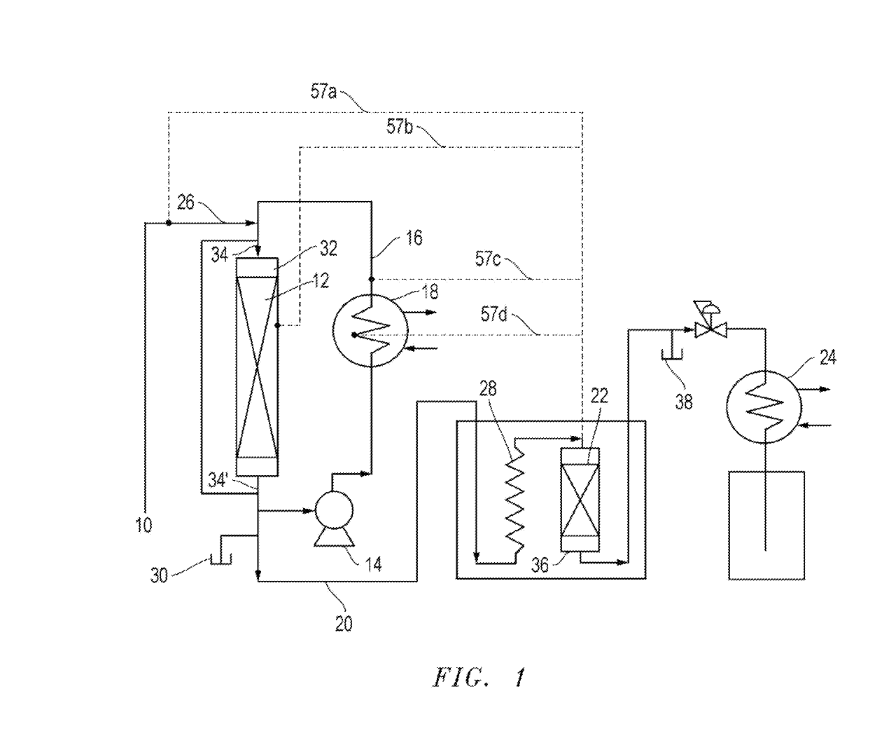

FIG. 1 is a schematic diagram of an adaptation of the two stage bench scale system used in Examples 4-6. Examples 4-6 were not technically run as a continuous process, for reasons discussed in more detail in the Examples. FIG. 1 is adapted to illustrate such a system for use in a continuous process

Referring to FIG. 1, by way of example, a cleavage reactor 12 is a column reactor packed with solid catalyst, and a decomposition reactor 22 is a plug flow column A cleavage feed comprising phenylalkyl hydroperoxides 10 is fed to a cleavage reactor 12 where the cleavage feed is exposed to cleavage conditions. In FIG. 1, the cleavage feed 10 is added to a recycle loop 16 at point 26.

The temperature in the cleavage reactor 12 is controlled by any suitable means. In the illustrated embodiment, the temperature is controlled by regulating the steam to the heat exchanger 18, as well as by the feed rate and the recycle rate.

A ketone feed can originate from any suitable source. In one embodiment, ketone feed is recycled from the decomposition reactor 22 to the cleavage reactor 12 at any suitable location. In one embodiment, a ketone feed 57a is introduced into the cleavage feed 10. In another embodiment, ketone feed 57b is introduced directly into the cleavage reactor 12. In another embodiment, ketone feed 57c is introduced into recycle loop 16. In yet another embodiment, ketone feed 57d is introduced via the heat exchanger 18 by a feed line which may be introduced into the condensing column, or the column containing the cleavage reaction mixture.

In the embodiment of FIG. 1, the cleavage product is transported from the cleavage reactor 12 via line 20 to the decomposition reactor 22, where the decomposition feed is exposed to solid decomposition catalyst under decomposition conditions. Referring to FIG. 1, the decomposition product is subjected to heat exchange, suitably by passing through a heat exchanger 24. The heat exchanger 24 may be any commercially available heat exchanger capable of handling the decomposition product. Examples include, but are not necessarily limited to a simple shell and tube heat exchanger.

The reaction product from the cleavage reactor 12 is fed via line 20 to the decomposition reactor 22 operating in the plug flow mode. The reaction product from the cleavage reactor 12 is heated to the desired temperature in a heat exchanger 28 and the temperature at the top of the decomposition reactor 22 is adjusted to maximize the conversion of dimethylbenzyl alcohol and dicumylperoxide to phenol, acetone, and α-methylstyrene. In this embodiment, solid catalyst is packed in the cleavage column 12. In one embodiment, solid catalyst also is packed in the decomposition reactor 22.

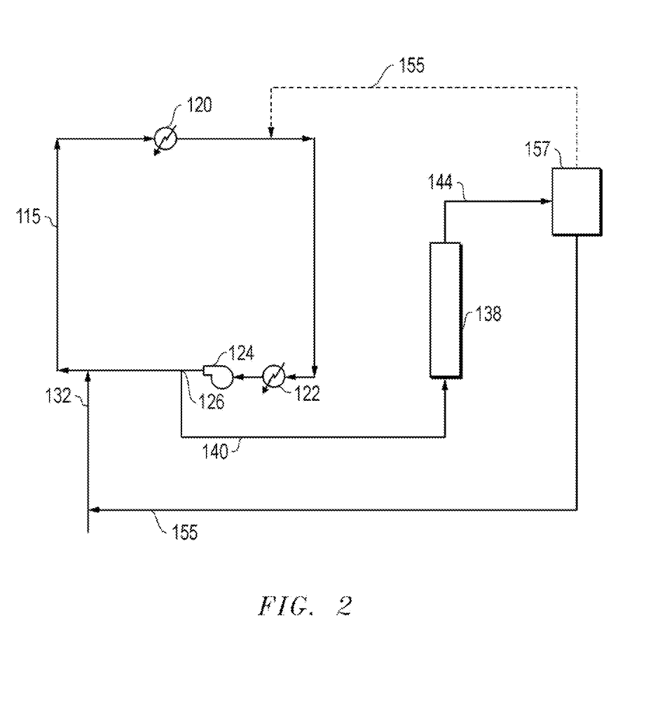

FIG. 2 illustrates another embodiment of a reactor system for practicing the present method. In this embodiment the cleavage reactor 115 is a pipeline loop reactor. The cleavage reactor 115 comprises one or more heat exchangers 120, 122 at appropriate locations to provide cooling sufficient to maintain the cleavage reaction mixture at the cleavage reaction temperature. A pump 124 is installed in the pipeline loop to provide for recirculation of a recycle flow of the cleavage reaction mixture, including catalyst, through the cleavage reactor 115. The cleavage reaction product 140, is withdrawn from the pipeline loop reactor at a withdrawal point 126 located a short distance upstream of the feed point 128 for the cleavage reactor feed 132.

Ketone feed 155 is fed into the cleavage reactor 115 at any suitable location. In one embodiment, a ketone feed 155 is fed to the first cleavage reactor feed 132. In another embodiment, indicated in dotted lines, a ketone feed 155 is fed directly to the cleavage reactor 115. Alternately, the ketone feed 155 is fed via the heat exchanger 120 and/or 122 by a feed line introduced into the condensing column In one embodiment, ketone feed 155 is recycled from a cleavage and/or decomposition product separation zone 157.

In one embodiment, the cleavage reaction product 140 is fed to a decomposition reactor 138, preferably a once through plug flow reactor, to produce a decomposition product 144. In one embodiment, the decomposition reactor 138 is packed with solid catalyst. The decomposition reactor 138 is operated at decomposition conditions effective to produce a decomposition product 144. The decomposition product 144 is withdrawn from the decomposition reactor 138 and passed to additional stages for recovering the decomposition products.

Optimum cleavage conditions and decomposition conditions may be established by adjusting various parameters. The conditions will vary with the number of stages, the number of reactors, and the type of reactor(s) used. For example, where the reactor is a packed bed reactor, the temperature and catalyst bed size may be varied to achieve maximum yield of hydroxybenzene and AMS. Persons of ordinary skill in the art will be able to establish optimum conditions using a particular reactor system, catalyst, and feed.

### The Decomposition Catalyst

Any solid catalyst having sufficient acidity to catalyze the decomposition reaction may be used. Preferred solid catalysts include, for example: acid clays, heteropolyacids, acid treated titania, and catalysts comprising one or more oxidized metals.

Examples of suitable acid clays include, but are not necessarily limited to kaolinite, attapulgite, montmorillonite, and cloisite clays.

Examples of suitable heteropolyacids include, but are not necessarily limited to tungstates, molybdates, vanadates, and combinations thereof. A specific example is 12-tungstophosphoric acid on a suitable support. Suitable supports include, for example, silica, alumina, titania, zirconia, and combinations thereof. Such heteropolyacids are described in U.S. Pat. No. 4,898,995, incorporated herein by reference.

Examples of suitable acid treated titania include, for example, titania treated with acid selected from the group consisting of fluorophosphoric acid, hydrofluoric acid, phosphoric acid, and combinations thereof. Such acid treated titanias include, for example, acidic polyanions comprising 12 WO₆ octahedra surrounding a PO₄ tetrahedron (Keggin primary structure).

Other examples of suitable solid catalysts include, for example, zeolite beta and Constraint Index 1-12 zeolites. See U.S. Pat. No. 6,410,804; U.S. Pat. No. 4,490,565; and, U.S. Pat. No. 4,490,566, each of which is incorporated herein by reference.

To the extent that the following catalysts are present as a solid under the decomposition conditions, the following are suitable for use as decomposition catalyst: faujasite, described in EP-A-492807; smectite clays, described in U.S. Pat. No. 4,870,217; ion exchange resins having sulfonic acid functionality, to the extent not soluble in the reaction mixture; sulfated oxidized transition metals such as sulfated zirconia together with oxidized iron or oxidized iron and oxidized manganese, as described in U.S. Pat. No. 6,169,216; mixed oxidized forms of cerium and a Group IVB metal, e.g., zirconium, described in U.S. Pat. No. 6,297,406; non-soluble rhenium compound, described in U.S. Pat. No. 4,173,587; zeolite crystals wherein a portion of the silicon atoms in the crystal lattice of silica is replaced by Al and B, where the zeolite crystals are bonded to each other by a siliceous bonding agent which allows the catalyst to assume the shape of mechanically stable microspheres, described in U.S. Pat. No. 4,743,573 and U.S. Pat. No. 4,849,387; SbF₅ and graphite, described in U.S. Pat. No. 4,487,970; boron phosphate, described in U.S. Pat. No. 4,480,141; fluorine-containing acidic compound on an inert support, described in U.S. Pat. No. 4,876,397; solid Lewis acid catalysts such as tin (II) chloride and tin (IV) fluoride, described in U.S. Pat. No. 4,267,380; composite of silica and alumina, described in U.S. Pat. No. 3,305,590; the metal complexes described in U.S. Pat. No. 4,262,153 and U.S. Pat. No. 3,928,477; acidic montmorillonite silica alumina clay, modified with a compound selected from the group consisting of a heteropoly acid, or the inorganic salt of zirconium, titanium and aluminum, described in U.S. Pat. No. 4,898,987; acidic smectite clays described in U.S. Pat. No. 4,870,217; gold, silver, copper or a sol-gel compound containing particular combinations of iron, nickel, chromium, cobalt, zirconium, tantalum, silicon, magnesium, niobium, aluminum, and titanium wherein certain of those metals are combined with an oxide, such as an inorganic matrix of hydroxides or oxides, optionally on a suitable support, described in U.S. Pat. No. 6,284,927; and, heterogeneous solid catalysts produced by calcining a source of a Group IV oxidized metal with a source of an oxyanion of a Group VIB metal, described in U.S. Pat. No. 6,269,215. Each of the foregoing references is incorporated herein by reference.

The solid catalysts have sufficient acidity to catalyze the decomposition of the di(phenylalkyl) peroxide(s) at temperatures of about 90° C. or more. In one embodiment, the catalysts are effective to catalyze the decomposition of the di(phenylalkyl)peroxide(s) at temperatures at about 50° C. or more.

Suitable oxidized metals for the decomposition catalyst are those in which the metal is selected from the following groups of the Periodic Table of the Elements: Group IB, IIB, IIIB, IVA, IVB, VA, VB, VIB, VIIB, VIIIB When the Periodic Table of the Elements is referred to herein, the source of the Periodic Table is: The Merck Index (12^{th} Ed. 1996). Examples of suitable oxidized metals include, for example, oxidized metals wherein the metal is selected from the group consisting of copper, silver, gold, zinc, cerium, titanium, zirconium, hafnium, germanium, tin, lead, vanadium, niobium, tantalum, antimony, bismuth, arsenic, chromium, molybdenum, tungsten, manganese, rhenium, and combinations thereof.

In a preferred embodiment, the decomposition catalyst comprises a combination of oxidized metals. Where the oxidized metal is an oxidized transition metal, the oxidized metal may be sulfated. Catalysts which are effective to cleave phenylalkyl hydroperoxides also should be effective to decompose di(phenylalkyl)peroxides.

Suitable catalysts for cleaving phenylalkyl hydroperoxides (cleavage catalysts), which also should cleave di(phenylalkyl)peroxides, include a combination of oxidized metals wherein the metals comprise: (i) a first metal selected from the group consisting of tin, iron, zinc, bismuth, cerium, and combinations thereof, and (ii) a second metal selected from the group consisting of zirconium, antimony, titanium, tungsten, and combinations thereof. In a preferred embodiment, (i) the first metal is selected from the group consisting of tin, zinc, and cerium, and (ii) the second metal is selected from the group consisting of tungsten and zirconium. In specific embodiments, the solid catalyst comprises oxidized forms of a combination of metals selected from the group consisting of: tungsten and tin; tungsten and iron; tungsten and cerium; tungsten and bismuth; tungsten and zinc; zirconium and tin; and, antimony and tin.

In one embodiment, the decomposition catalyst comprises a combination of oxidized tungsten and oxidized forms of one or more metal selected from the group consisting of tin, zinc, and cerium. In one embodiment, the decomposition catalyst comprises a combination of oxidized tungsten and oxidized cerium. In one embodiment, the decomposition catalyst comprises a combination of oxidized tungsten and oxidized tin. In one embodiment, the decomposition catalyst comprises a combination of oxidized tungsten and oxidized zinc.

Some oxidized metals have a tendency to slowly dissolve in the cleavage and/or decomposition reaction mixture. Other oxidized metals have less of a tendency to dissolve in the reaction mixture. Catalysts comprising a combination of oxidized tin and oxidized zirconium have less of a tendency to dissolve in the reaction mixture. In one embodiment, oxidized tungsten is replaced by oxidized zirconium. In one embodiment, the solid catalyst comprises oxidized tin and oxidized zirconium. In one embodiment, the catalyst comprises a support comprising about 10 wt. % zirconium and about 5 wt. % tin (IV), based on the total weight of the catalyst, both in oxidized form.

### Preparation of Heterogeneous Solid Catalyst

The following procedures may be used to prepare solid catalyst comprising one or more oxidized metals. These catalysts may be used as cleavage catalyst and/or as decomposition catalyst.

The solid catalyst may consist of 100 wt. % oxidized metal, preferably a combination of oxidized metals. Where the catalyst is 100% oxidized metal, the catalyst may be made according to the methods described in U.S. Pat. No. 6,169,215, incorporated herein by reference. Generally, that method involves calcining a source of a first oxidized metal with a source of an oxyanion of a second metal at a temperature of 400° C. or more.

The use of a support is preferred, particularly where the use of a support reduces the cost of the catalyst. A variety of supports are useful. By way of example, suitable supports include but are not necessary limited to silica, alumina, silica-alumina, titania, zirconia, zeolites, and acidic clay, including pillared clay. Where the support comprises one or more zeolitic material(s), suitable zeolites include, but are not necessarily limited to zeolite beta and zeolites having a Constraint Index of from 1 to 12.

Supported oxidized metals may be made using a variety of methods and may comprise a variety of structures. Suitable methods of preparation include, but are not necessarily limited to impregnation, coimpregnation, including single or multiple impregnations, precipitation, including single or multiple precipitations, physical admixture or any other suitable method. The method employed will depend on the solubility of the source of metal and the conditions required to convert the source to the metal. In a preferred embodiment, supported catalyst comprising a combination of metals is prepared by precipitation of sources of a combination of metals onto the support, separately or in the same procedure. Suitable sources of metals for precipitation include, but are not necessarily limited to metal nitrates, metal chlorides, metal acetates, metal sulfates, and metal ammonium salts, etc. Suitable water soluble sources of zirconium include, but are not necessarily limited to zirconyl chloride, zirconyl nitrate, zirconium tetraacetate, and combinations thereof.

Where the source of metal is a metal chloride, it is convenient to hydrolyze the metal chloride in the presence of acid or base before calcination of the impregnated support. Substantially any acid or base effective to hydrolyze the metal chloride may be used. In the examples, ammonium hydroxide was used as the base. Dilute nitric acid was used as the acid.

After deposition of the sources of a combination of metals onto the support, the material is dried and calcined at a temperature of 400° C. or more, preferably 500° C. or more, typically from 500° C. to 1000° C., for a period of 2 hours to 30 hours.

Preferably, the catalyst comprises about 40 wt. % or less, suitably about 5 wt. % or more, of a combination of metals based on the total weight of the catalyst, including the oxidized metals and any support, as measured by elemental analysis. In one embodiment, the catalyst comprises from about 2 wt. % to about 20 wt. % of each of two oxidized metals, based on the total weight of the catalyst, as measured by elemental analysis.

After calcination, the catalyst comprises a combination of a first amount of a first oxidized metal and a second amount of a second oxidized metal. The combination has sufficient acidity to cleave the phenylalkyl hydroperoxide and/or to decompose the di(phenylalkyl)peroxide. In one embodiment, the acidity of the combination is greater than that of a mixture formed by separately oxidizing the first metal and the second metal and subsequently mixing the first amount of the oxidized first metal with the second amount of the oxidized second metal.

Without wishing to be bound by theory, it is believed that the acidity of the combination of oxidized metals is increased by calcining the metals together at the same time. Again, without limiting the application to a particular structure, it has been theorized that superacids are formed when sulfates and possibly tungstates react with hydroxides or oxides of certain metals. It is believed that these superacids have the structure of a bidentate sulfate or tungstate ion coordinated to the metal. However, the particular structure of the catalytically active site has not been confirmed.

The invention will be more clearly understood with reference to the following Examples, which are provided by way of illustration only.

### EXAMPLES

### Synthesis of Solid Catalysts

Examples 1-3 illustrate the preparation of solid catalysts for use in the cleavage and/or decomposition processes according to the present invention.

### Example 1

20 grams of 99 wt. % silica obtained from CRI International (Houston, Tex., USA) (“CRI”) was impregnated with a solution containing 8.8 grams of tin tetrachloride in 15 ml of methylene chloride. The solution was mixed with the silica for 20 minutes. The solvent was evaporated and the resultant solid was immersed in 4 molar, 14 wt. % ammonium hydroxide solution. The mixture was allowed to stand overnight. The mixture was washed with deionized water until neutral. The solid was dried in a vacuum oven at 80-120° C. overnight. The dried, tin-impregnated silica was impregnated with a solution containing 1.35 grams of ammonium metatungstate in 20 grams of deionized water. The resulting metal impregnated silica was dried at 80-120° C. and calcined at 1000° C. in flowing air for 3 hours. The catalyst contains approximately 16 wt. % tin and approximately 3.8 wt. % tungsten by the mode of synthesis.

### Example 2

7.6 grams of Ce (NO₃)₃.6H₂O were dissolved with stirring in 20 grams of deionized water. 20 grams of silica extrudate obtained from CRI was impregnated with the cerium solution and aged at room temperature overnight. The Ce-impregnated silica was then dried and the solvent evaporated in an oven at 150° C. for 3 hours. The dried Ce-impregnated catalyst was impregnated with a solution containing 1.65 grams ammonium metatungstate in 20 ml of deionized water and aged for 1 hour. The resulting impregnated catalyst was dried and the solvent evaporated in the oven at 150° C. for 1 hour and calcined at 1000° C. for 3 hours. The catalyst contains approximately 10 wt. % cerium and approximately 5 wt. % tungsten by the mode of synthesis.

### Example 3

146.05 grams of Zn(O₂CCH₃)₂.2H₂O were dissolved with stiffing in 28 grams of deionized water. 30 grams of silica extrudate obtained from CRI was impregnated with the solution and allowed to age for 1 hour. The Zn-impregnated silica was dried and the solvent evaporated in an oven at 150° C. for 3 hours. The dried Zn-impregnated silica was impregnated with a solution containing 5.06 grams of ammonium metatungstate in 30 grams of deionized water. The catalyst was aged for 1 hour and dried and the solvent evaporated in the oven at 150° C. for 3 hours and calcined at 800° C. for 3 hours. The catalyst contains approximately 48.9 wt. % zinc and approximately 4.3 wt. % tungsten by the mode of synthesis.

### Cleavage and Decomposition

The following examples used a continuous bench scale unit to perform the process of the present invention. The bench scale unit is schematically illustrated in FIG. 1.

Referring to FIG. 1, the cleavage reactor 12 was a column reactor and the decomposition reactor 22 was a plug flow column. The total volume of reaction mixture in the cleavage reactor 12 was approximately 250 ml.

Cumene hydroperoxide 82-86 wt. % was added to the recycle loop 16 at point 26 using a dual set of 250 ml capacity Isco pumps 14 equipped for continuous and accurate feed control. The temperature in the cleavage reactor 12 was controlled by regulating the steam to the heat exchanger 18. Once the reaction started, the reaction was controlled by heat exchanger 18, feed rate and recycle rate. The temperature was measured across the cleavage reactor 12 at 34-34′. The reaction product from the cleavage reactor 12 was fed via line 20 to the decomposition reactor 22 operating in the plug flow mode. The reaction product from the cleavage reactor 12 was heated to the desired temperature in a heat exchanger 28 and the temperature at the top of the decomposition reactor 22 was adjusted to maximize the conversion of dimethylbenzyl alcohol and dicumylperoxide to phenol, acetone, and α-methylstyrene.

In order to analyze the cleavage product and to adjust reaction conditions, the cleavage product was collected at a first sampling port 30. The sampled cleavage product was analyzed once a day using high performance liquid chromatography (HPLC). After a large volume of cleavage product samples were collected over a period of several days, the collected samples were well mixed. About 500 ml. of the well-mixed, combined samples were fed to an Isco pump in fluid communication with line 20 and passed through the decomposition reactor 22. This allowed for varying conditions and demonstrated that acceptable decomposition product could be made from a variety of first stage feeds. The decomposition product was sampled at a second sampling port 38 and analyzed once a day using high performance liquid chromatography (HPLC).

Although the bench scale process was not run as a continuous process, as depicted in FIG. 1, an industrial process normally would be run as a continuous process.

### Example 4

### Cleavage

100 g of the tin/tungsten catalyst of Example 1 was packed in the cleavage column 12. The feed to the cleavage column 12 comprised 86 wt. % cumene hydroperoxide containing 9.6 wt. % cumene, 3.6 wt. % dimethylbenzylalcohol, 0.4 wt. % acetophenone, and 0.2 wt. % dicumylperoxide. The feed rate was 100 g total feed/hour and the recycle rate through line 16 was 4000 g/hour. The temperature near the top 32 of the cleavage column 12 was 60° C. The ΔT, or temperature difference across the column (34-34′ in FIG. 1) was 10-11° C. The cleavage product (line 20) was sampled once a day via sampling port 30. The following Table shows the analysis of minor components of the cleavage product for 138 hours. (The remainder being acetone and phenol). The amount of acetophenone remained constant at 0.4% wt.

| | | cumene | | | | | dimers of |
|---|---|---|---|---|---|---|---|
| | dimethylbenzyl | hydro- | | α-methyl- | | dicumyl- | α-methyl- |
| Time | alcohol | peroxide | cumylphenol | styrene | cumene | peroxide | styrene |
| hrs. | wt % | wt % | wt % | wt % | wt % | wt % | wt % |
| 0 | 0.26 | 0.0 | 0.27 | 1.83 | 9.47 | 1.13 | 0.3 |
| 18 | 0.26 | 0.72 | 0.40 | 1.54 | 8.39 | 1.35 | 0.3 |
| 42 | 0.36 | 1.53 | 0.31 | 1.47 | 8.76 | 2.18 | 0.2 |
| 66 | 0.50 | 2.59 | 0.23 | 1.42 | 9.82 | 3.24 | 0.1 |
| 90 | 0.60 | 3.73 | 0.17 | 1.23 | 10.00 | 3.88 | 0.1 |
| 114 | 0.61 | 3.79 | 0.13 | 0.98 | 9.15 | 3.84 | 0.1 |
| 138 | 0.61 | 3.68 | 0.21 | 1.48 | 10.11 | 3.84 | 0.1 |

### Decomposition

The cleavage product was collected and passed through the decomposition reactor 22 containing 100 g of the oxidized tin/tungsten catalyst, produced as described in Example 1. The decomposition reactor 22 was run at various feed rates, at various top of the column temperatures, and at a temperature at the bottom 36 of the decomposition reactor of 115-117° C. to decompose the remaining cumene hydroperoxide and dicumyl peroxide. The feed to the decomposition reactor comprised 0.45 wt. % acetophenone, 0.5 wt. % dimethylbenzylalcohol, 0.82 wt. % cumene hydroperoxide, 0.3 wt. % cumylphenol, 1.4 wt. % α-methylstyrene, 11 wt. % cumene, 2.7 wt. % dicumylperoxide with the remainder being acetone and phenol.

The decomposition product was sampled at the sampling port 38 and analyzed by HPLC. The following results were obtained at varying conditions:

CONDITIONS

| | | | |
|---|---|---|---|
| Top, ° C. | 42.8 | 55.6 | 60.7 |
| Bottom, ° C. | 116.1 | 116.7 | 117.4 |
| Feed Rate, g/hr | 60 | 100 | 120 |

ANALYSIS (wt. %)

| Sample | Feed | | | |
|---|---|---|---|---|
| acetophenone | 0.45 | 0.60 | 0.56 | 0.56 |
| dimethylbenzyl alcohol | 0.51 | 0.11 | 0.14 | 0.17 |
| cumene hydroperoxide | 0.82 | 0 | 0.0 | 0.00 |
| cumylphenol | 0.33 | 0.69 | 0.53 | 0.47 |
| α-methylstyrene | 1.41 | 2.86 | 2.64 | 2.53 |
| cumene | 10.98 | 11.13 | 10.98 | 11.01 |
| dicumylperoxide | 2.71 | 0.38 | 0.97 | 1.24 |
| dimers of α-methylstyrene | 0.01 | 0 | 0.03 | 0.01 |
| α-methylstyrene selectivity | | 0.82 | 0.75 | 0.73 |

The foregoing results demonstrate that the oxidized tin/tungsten catalyst was effective at a bottom column temperature of from 115-117° C. and a decomposition feed rate of 60 g/hr to treat a decomposition feed containing about 3 wt. % or less (specifically 2.71 wt. %) dicumylperoxide to produce a decomposition product containing 0.5 wt. % or less dicumylperoxide and a selectivity to α-methyl styrene of 0.82, based on the components in the decomposition feed that could theoretically produce AMS.

### Example 5

The cleavage product was collected and passed through the decomposition reactor 22 containing 100 g of the oxidized cerium/tungsten catalyst, prepared as described in Example 2. The decomposition feed was passed through the decomposition column at various feed rates, at various reactor top temperatures, and at a decomposition reactor bottom temperature of 115-117° C. to decompose the remaining cumene hydroperoxide and dicumyl peroxide.

The following results were obtained at varying conditions:

CONDITIONS

| | | | | | |
|---|---|---|---|---|---|
| Top, ° C. | 63.2 | 55.9 | 43.7 | 36.2 | 35.1 |
| Bottom, ° C. | 115.7 | 116.1 | 116.1 | 116.6 | 116.1 |
| Feed Rate, g/hr | 120 | 100 | 60 | 45 | 30 |

ANALYSIS (wt. %)

| Sample | Feed | | | | | |
|---|---|---|---|---|---|---|
| acetophenone | 0.45 | 0.52 | 0.42 | 0.46 | 0.46 | 0.46 |
| dimethylbenzyl alcohol | 0.51 | 0.15 | 0.13 | 0.12 | 0.16 | 0.13 |
| cumene hydroperoxide | 0.82 | 0.20 | 0.00 | 0 | 0 | 0 |
| cumylphenol | 0.33 | 0.51 | 0.41 | 0.52 | 0.37 | 0.4 |
| α-methylstyrene | 1.41 | 2.56 | 2.10 | 2.44 | 2.1 | 2.26 |
| cumene | 10.98 | 10.92 | 9.28 | 10.40 | 9.79 | 9.42 |
| dicumylperoxide | 2.71 | 1.01 | 0.88 | 0.66 | 0.53 | 0.48 |
| dimers | 0.01 | 0.02 | 0.02 | 0.03 | 0.02 | 0.02 |
| selectivity for α-methylstyrene | | 0.71 | 0.74 | 0.77 | 0.78 | 0.80 |

The foregoing results demonstrate that the oxidized cerium/tungsten catalyst was effective at a bottom column temperature of from 115-117° C. and a decomposition feed rate of 30 g/hr to treat a decomposition feed containing about 3 wt. % or less (specifically 2.71 wt. %) dicumylperoxide to produce a decomposition product containing 0.5 wt. % or less dicumylperoxide and a selectivity to α-methyl styrene of 0.80, based on the components in the decomposition feed that could theoretically produce AMS.

### Example 6

A different batch of combined samples of cleavage product was collected and passed through the decomposition reactor 22 containing 100 g of the oxidized zinc/tungsten catalyst, prepared as described in Example 3. The decomposition feed was passed through the decomposition column at various feed rates and at a decomposition reactor bottom temperature of 115-117° C. to decompose the remaining cumene hydroperoxide and dicumyl peroxide.

The following results were obtained at varying conditions:

CONDITIONS

| | | | |
|---|---|---|---|
| Top, ° C. | 61.3 | 58 | 44.6 |
| Bottom, ° C. | 115.7 | 116.8 | 117.4 |
| Feed Rate, g/hr | 120 | 100 | 60 |

ANALYSIS (wt. %)

| Sample | Feed | | | |
|---|---|---|---|---|
| acetophenone | 0.39 | 0.44 | 0.45 | 0.47 |
| dimethylbenzyl alcohol | 0.32 | 0 | 0 | 0 |
| cumene hydroperoxide | 0.39 | 0 | 0.00 | 0 |
| cumylphenol | 0.69 | 1.00 | 0.94 | 0.94 |
| α-methylstyrene | 1.33 | 1.72 | 1.80 | 1.90 |
| cumene | 9.22 | 9.26 | 9.47 | 9.64 |
| dicumylperoxide | 2.76 | 0.32 | 0.40 | 0.39 |
| dimers | 0.04 | 0.08 | 0.07 | 0.06 |
| selectivity for α-methylstyrene | | 0.69 | 0.70 | 0.72 |

The foregoing results demonstrate that the oxidized zinc/tungsten catalyst was effective at bottom column temperatures of from 115-117° C. and at a decomposition feed rate of 100 g/hr to treat a decomposition feed containing about 3 wt. % or less (specifically 2.76 wt. %) dicumylperoxide to produce a decomposition product containing 0.5 wt. % or less dicumylperoxide and a selectivity to α-methyl styrene of 0.70, based on the components in the decomposition feed that could theoretically produce AMS. The foregoing also demonstrates that, when the feed rate was reduced to 60 g/hr, the selectivity to α-methyl styrene increased to 0.72, based on the components in the decomposition feed that could theoretically produce AMS.

Persons of ordinary skill in the art will recognize that many modifications may be made to the foregoing description. The embodiments described herein are meant to be illustrative only and should not be taken as limiting the invention, which will be defined in the claims.

1. A method for decomposing di(phenylalkyl) peroxide comprising feeding a decomposition feed comprising di(phenylalkyl)peroxide to a reactor containing solid decomposition catalyst under decomposition conditions which decompose the di(phenylalkyl)peroxide to produce hydroxybenzene and phenylalkene, wherein the solid decomposition catalyst comprises a combination of oxidized metals wherein the metals comprise:
a first metal selected from the group consisting tin, iron, zinc, bismuth, cerium, and combinations thereof, and
a second metal selected from the group consisting of zirconium, antimony, titanium, tungsten, and combinations thereof.

2. The method of claim 1 wherein the decomposition feed comprises from 0.5 wt. % to 5 wt. % di(phenylalkyl)peroxide and the decomposition conditions are effective to convert 80 wt. % or more of the di(phenylalkyl)peroxide in the decomposition feed to phenylalkene and hydroxybenzene.

3. The method of claim 1 wherein the decomposition conditions are effective to produce a decomposition product comprising from 0.05 wt. % to 0.1 wt. % di(phenylalkyl)peroxide, based on the total weight of the decomposition product.

4. The method of claim 1 wherein the decomposition product comprises 0.5 wt. % or less di(phenylalkyl)peroxide.

5. The method of claim 2 wherein the decomposition conditions comprise a decomposition temperature of from 110° C. to 120° C.

6. (canceled)

7. (canceled)

8. (canceled)

9. The method of claim 1 wherein the solid decomposition catalyst comprises a combination of oxidized tungsten and oxidized other metal wherein the other metal is selected from the group consisting of tin, cerium, zinc, and combinations thereof.

10. The method of claim 1 wherein the solid decomposition catalyst comprises a combination of oxidized zirconium and oxidized other metal, wherein the other metal is selected from the group consisting of tin, cerium, zinc, and combinations thereof.

11. A method for decomposing di(phenylalkyl) peroxide comprising:
subjecting a cleavage feed comprising phenylalkyl hydroperoxide in the presence of cleavage catalyst to cleavage conditions which cleave the phenylalkyl hydroperoxide and produce a cleavage product comprising the di(phenylalkyl)peroxide; and,
subjecting a decomposition feed comprising the cleavage product to solid decomposition catalyst under decomposition conditions which decompose the di(phenylalkyl)peroxide and produce hydroxybenzene and phenylalkene, wherein the solid decomposition catalyst comprises a combination of oxidized metals wherein the metals comprise:
a first metal selected from the group consisting tin, iron, zinc, bismuth, cerium, and combinations thereof, and
a second metal selected from the group consisting of zirconium, antimony, titanium, tungsten, and combinations thereof.

12. The method of claim 11 wherein the cleavage catalyst is solid cleavage catalyst.

13. The method of claim 12 wherein the decomposition conditions produce a selectivity to phenylalkene of 0.70 or more, based on the components in the decomposition feed that can theoretically produce the phenylalkene.

14. (canceled)

15. (canceled)

16. The method of claim 11 wherein the decomposition feed comprises from 0.5 wt. % to 5 wt. % di(phenylalkyl)peroxide and the decomposition conditions are effective to convert 80 wt. % or more of the di(phenylalkyl)peroxide in the decomposition feed to phenylalkene and hydroxybenzene.

17. The method of claim 11 wherein the decomposition conditions are effective to produce a decomposition product comprising from 0.05 wt. % to 0.1 wt. % di(phenylalkyl)peroxide, based on the total weight of the decomposition product.

18. (canceled)

19. (canceled)

20. (canceled)

21. The method of claim 17 wherein
the cleavage conditions produce a cleavage product further comprising hydroxybenzene and ketone and comprise a cleavage temperature of from 50° C. to 90° C.; and,
the decomposition conditions comprise a decomposition temperature of from 90° C. to 170° C.

22. (canceled)

23. (canceled)

24. (canceled)

25. (canceled)

26. The method of claim 21 wherein the cleavage conditions and the decomposition conditions occur in different reactors.

27. (canceled)

28. The method of claim 11 wherein one or both of the solid cleavage catalyst and the solid decomposition catalyst comprise a combination of oxidized metals wherein the metals comprise:
a first metal selected from the group consisting tin, iron, zinc, bismuth, cerium, and combinations thereof, and
a second metal selected from the group consisting of zirconium, antimony, titanium, tungsten, and combinations thereof.

29. (canceled)

30. (canceled)

31. A method for decomposing di(phenylalkyl) peroxide comprising:
feeding a cleavage feed comprising phenylalkyl hydroperoxide to a first column packed with solid cleavage catalyst comprising a combination of oxidized metals and subjecting the cleavage feed to cleavage conditions which cleave the phenylalkyl hydroperoxide and produce a cleavage product comprising the di(phenylalkyl)peroxide; and,
feeding the decomposition feed comprising the cleavage product to a second column packed with solid decomposition catalyst comprising a combination of oxidized metals under decomposition conditions which decompose the di(phenylalkyl)peroxide and produce hydroxybenzene and phenylalkene, wherein the solid decomposition catalyst comprises a combination of oxidized metals wherein the metals comprise:
a first metal selected from the group consisting tin, iron, zinc, bismuth, cerium, and combinations thereof, and
a second metal selected from the group consisting of zirconium, antimony, titanium, tungsten, and combinations thereof.

32. (canceled)

33. (canceled)

34. (canceled)

35. The method of claim 31 wherein the decomposition feed comprises from 0.5 wt. % to 5 wt. % di(phenylalkyl)peroxide and the decomposition conditions are effective to convert 80 wt. % or more of the di(phenylalkyl)peroxide in the decomposition feed to phenylalkene and hydroxybenzene.

36. (canceled)

37. (canceled)

38. The method of claim 31 wherein
the cleavage conditions produce a cleavage product further comprising hydroxybenzene and ketone and comprise a cleavage temperature of from 50° C. to 90° C.; and,
the decomposition conditions comprise a decomposition temperature of from 90° C. to 170° C.

39. (canceled)

40. (canceled)

41. (canceled)

42. (canceled)

43. (canceled)

44. (canceled)

45. (canceled)

46. The method of claim 31 wherein
(a) the cleavage conditions comprise a top column temperature from 35° C. to 60° C. and a cleavage feed rate of from 0.3 to 1.0 grams of feed per gram of catalyst per hour; and
(b) the decomposition conditions comprise a bottom column temperature of from 90° C. to 126° C. and a decomposition feed rate of from 0.3 gram to 1 gram of feed per gram of catalyst per hour.
